(19) United States
(12) Patent Application Publication
(10) Pub. No.: US 2026/0191401 A1
(43) Pub. Date: Jul. 9, 2026

(54) MEDICAL SYSTEMS AND DEVICES WITH A CUSTOMIZABLE HANDLE

(71) Applicant: Boston Scientific Scimed, Inc., Maple Grove, MN (US)

(72) Inventors: John SUBASIC, Sutton, MA (US); Niraj Prasad RAUNIYAR, Plymouth, MN (US)

(73) Assignee: Boston Scientific Scimed, Inc., Maple Grove, MN (US)

(21) Appl. No.: 19/441,153

(22) Filed: Jan. 6, 2026

Related U.S. Application Data
(60) Provisional application No. 63/743,299, filed on Jan. 9, 2025.

(51) Int. Cl. A61B 1/00

(52) U.S. Cl. CPC A61B 1/00147; A61B 1/00068; A61B 1/00119; A61B 1/00128; A61B 1/0014

(57) ABSTRACT

Medical devices useful to perform medical procedures are described. The medical device may include a handle with a hub and a port. The hub may define a lumen and include a proximal opening configured to receive a medical instrument. The port may be configured to couple to a source of suction, and the port may be in fluid communication with the lumen of the hub. The outer surface of the hub or the port may include a mounting interface. The handle may also include a grip accessory configured to releasably couple to the mounting interface. The grip accessory may include an opening or groove configured to receive a portion of a user's hand when the user grips the handle.

### CROSS-REFERENCE TO RELATED APPLICATIONS

This application claims the benefit of priority to U.S. Provisional Application No. 63/743,299, filed on Jan. 9, 2025, which is incorporated by reference herein in its entirety.

### TECHNICAL FIELD

Aspects of this disclosure generally relate to medical devices with handles that have custom features. In particular, this disclosure includes medical devices with handles that includes releasably attachable grip accessories.

### BACKGROUND

Medical devices such as scopes and sheaths are often inserted into a patient's body to perform medical procedures inside the body. These medical devices may include a handle providing the user control over navigation and/or other features of the medical device. Users may have varying grip preferences, techniques, and hand sizes, which can present challenges to adapt to different users. Additionally, as medical techniques in procedures evolve and become more specialized, the need for medical devices that may be tailored to individual preferences and procedural requirements continues to grow.

### SUMMARY

The disclosure includes medical devices useful to perform medical procedures. For example, the medical device may comprise a handle with a hub and a port. The hub may define a lumen and include a proximal opening configured to receive a medical instrument. The port may be configured to couple to a source of suction, the port being in fluid communication with the lumen of the hub. The outer surface of the hub or the port may include a mounting interface. The handle may also comprise a grip accessory configured to releasably couple to the mounting interface. The grip accessory may include an opening or groove configured to receive a portion of a user's hand when the user grips the handle.

According to some aspects, the mounting interface is a first mounting interface and one of the hub or the port includes a second mounting interface. The grip accessory may be a first grip accessory, and the medical device may further comprise a second grip accessory configured to releasably couple to the second mounting interface. In some aspects, the grip accessory(ies) is/are configured to releasably couple to both the first mounting interface and the second mounting interface. In at least one example, the hub includes the first mounting interface, and the port includes the second mounting interface. The mounting interface may include a plurality of engagement features configured to receive a plurality of engagement features of the grip accessory. In some aspects, the mounting interface may span an arc. Additionally, or alternatively, the port or the hub may include a vent, optionally wherein the mounting interface is proximate the vent. The vent may include a slider configured to cover and uncover an aperture of the port or the hub. The mounting interface may be a first mounting interface, and the grip accessory may be a first grip accessory, and the slider may include a second mounting interface configured to releasably couple to a second grip accessory. At least one of the hubs or the port may include a second mounting interface proximate the first mounting interface. In some aspects, the hub may include the first mounting interface and the second mounting interface. In some aspects, the port may include the first mounting interface and the second mounting interface. The hub may further comprise a third mounting interface. The medical device may further comprise a sheath defining a channel in fluid communication with the lumen and the port of the handle.

This disclosure also includes a medical device comprising a handle with a hub and a port, wherein the hub defines a lumen and includes a proximal opening configured to receive a medical instrument. The port may be configured to be coupled to a source of suction, the port being in fluid communication with the lumen of the hub. The outer surface of the hub or the port may include a first mounting interface and a second mounting interface. The handle may also comprise a first grip accessory configured to releasably couple to the first mounting interface and a second grip accessory configured to releasably couple to the second mounting interface. The first grip accessory and the second grip accessory may include an opening or groove configured to receive a portion of a user's hand when the user grips the handle.

According to some aspects, the handle may further comprise a vent including a slider configured to cover and uncover an aperture of the port or the hub, and the first mounting interface may be proximate the vent. In some examples, the first mounting interface is on the hub and spans an arc along the outer surface of the hub, and the second mounting interface is on the port and extends a length of the outer surface of the port. The first mounting interface may include a plurality of engagement features configured to releasably couple with corresponding engagement features on the first grip accessory.

This disclosure also includes a medical device that includes a handle comprising a hub, a port, and a vent. The hub may define a lumen and include a proximal opening configured to receive a medical instrument. The port may be configured to be coupled to a source of suction, the port being in fluid communication with the lumen of the hub. The vent may include a slider configured to cover and uncover an aperture of the port or the hub. The outer surface of the port may include a first mounting interface, and an outer surface of the slider may include a second mounting interface. The medical device may further comprise a first grip accessory configured to releasably couple to the first mounting interface, and a second grip accessory configured to releasably couple to the second mounting interface. According to some aspects, the hub may include a third mounting interface.

### BRIEF DESCRIPTION OF THE FIGURES

The accompanying drawings, which are incorporated in and constitute a part of this application, illustrate exemplary aspects that, together with the written description, explain the principles of this disclosure. The figures depict exemplary aspects according to this disclosure, as follows:

FIG. 1 shows a side view of an exemplary medical device with a handle and grip accessories, according to aspects of this disclosure;

FIG. 2 shows a side view of another exemplary medical device with a handle and grip accessories attached to the handle, according to aspects of this disclosure;

FIG. 3 shows a side view of another exemplary medical device with a handle and grip accessories attached to the handle, according to aspects of this disclosure; and

FIG. 4 shows a side view of another exemplary medical device with grip accessories attached to the handle, according to aspects of this disclosure.

### DETAILED DESCRIPTION

Particular aspects of the disclosure are described in greater detail below. The terms and definitions provided herein control, if in conflict with terms and/or definitions incorporated by reference. The terms “proximal” and “distal” are used herein to refer to the relative positions of the components of exemplary medical devices. As used herein, “proximal” refers to a position relatively closer to the exterior of the body or closer to a user or an operator (e.g., a physician or other medical professional) using the medical device. In contrast, “distal” refers to a position relatively further away from the operator using the medical device, or closer to the interior of the body.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Although aspiration sheaths and scopes such as ureteroscopes are referenced herein for illustration purposes, it will be appreciated that the disclosure encompasses any suitable medical device configured to help an operator to access and view internal body anatomy of a subject and/or to deliver suction, irrigation, and/or medical instruments such as, for example, biopsy forceps, graspers, baskets, snares, probes, scissors, capture/retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration sheaths, catheters, or any other suitable delivery device may be used in connection with the features described herein.

Aspects of the disclosure describe exemplary medical systems and devices that may enable customizable handle configurations through various grip accessories. Grip accessories comprise components removably attachable to one or more corresponding mounting interfaces of the handle of the medical device in order to facilitate grip. The mounting interface(s) are located on portions of the handle configured to support the palm and/or one or more digits of the user's hand to help the user hold and manipulate the handle. Additionally, the grip accessories may include various configurations and sizes, providing for a greater ergonomic and customizable experience for the user.

The medical devices herein may include a handle having a hub and a port. The outer surface of the hub and/or port may include one or more mounting interfaces as mentioned above. These mounting interfaces may include engagement features configured to securely attach and detach corresponding engagement features on one or more grip accessories. The grip accessories may include an opening or groove that can accommodate a portion of the user's hand while gripping the handle.

The medical devices may be configured to receive different grip accessories with assorted sizes and/or configurations, the grip accessories having compatible engagement features for the mounting interfaces at various positions on the handle. The handle's adaptability through releasable grip accessories may help medical professionals to adjust the handle to suit a desired hand position and orientation based on their preferences and procedural considerations, helping to enhance comfort, improve control, and increase procedural efficiency.

Reference will now be made in detail to examples to help illustrate aspects of the disclosure through the accompanying drawings. Wherever possible, the same reference numbers will be used throughout the drawings to refer to the same or like parts.

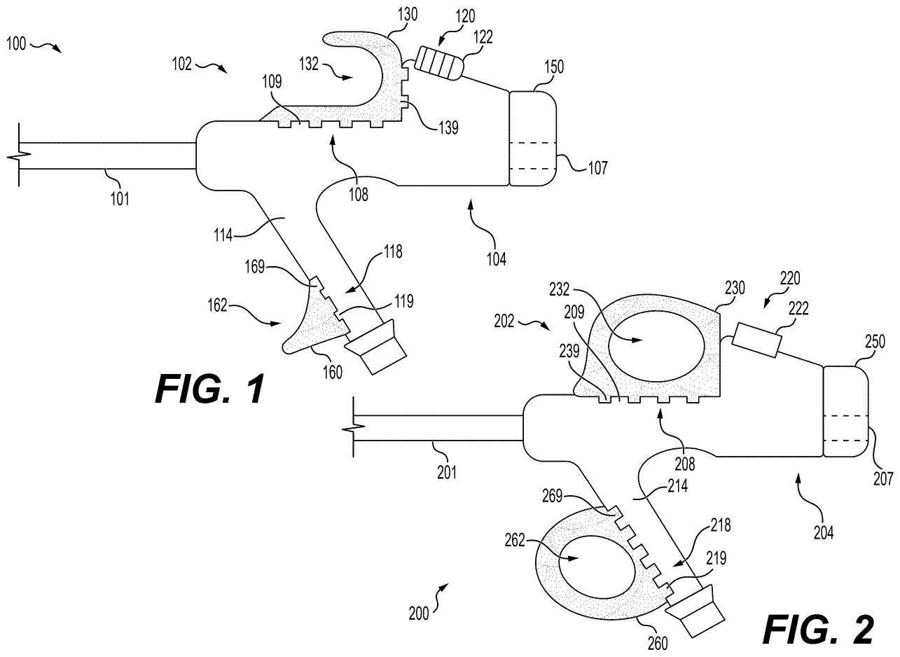

FIG. 1 depicts an exemplary medical device 100. Optionally, medical device 100 may be configured to receive a medical instrument such as a scope. The medical device 100 may be configured to help a user (e.g., medical professional) to deliver medical instruments to access internal areas of a subject's body, e.g., to perform medical diagnoses and/or treatments on the subject, with the ability to provide suction and/or irrigation. The medical device 100 includes a handle 102 that may be coupled to or integral with a sheath 101. Thus, for example, the handle 102 may be in fluid communication with a channel of the sheath 101.

The handle 102 includes a hub 104 that may define a proximal opening 107 to slidably receive a medical instrument such as a scope (e.g., ureteroscope, endoscope, hysteroscope, bronchoscope, cystoscope, or other scope or similar device) into a lumen defined by handle 102 (e.g., a lumen of hub 104). The proximal opening 107 may include a seal 150 configured to maintain a position of a medical instrument relative to medical device 100, forming a substantially airtight and/or watertight seal around the medical instrument received therein.

The handle 102 also includes a port 114 in fluid communication with the lumen of hub 104. Port 114 may extend along an axis transverse to the longitudinal axis of hub 104. Handle 102 may include features that help the user to control or adjust pressure through medical device 100 when port 114 is coupled to a vacuum source. As shown in FIG. 1, hub 104 includes a vent 120 configured to control suction pressure in medical device 100. For example, vent 120 may include an aperture within the wall of hub 104 and a slider 122 configured to cover and uncover the aperture of vent 120. Accordingly, a user may cover and uncover the aperture to control fluid communication with the external environment, such that suction pressure may be increased when the aperture is fully closed. In certain configurations, slider 122 may comprise a softer material (e.g., plastic) compared to the hub 104. A softer material may help slider 122 better conform to the outer surface of the hub 104 and/or port 114.

Medical device 100 also includes two grip accessories 130, 160 in this example. It will be understood that the medical devices herein may include only one grip accessory or more than two grip accessories. Each grip accessory 130, 160 may be removably attachable to the handle 102 and have features that help a user hold and manipulate the handle 102. For example, the grip accessory 130 and the grip accessory 160 may include respective grooves 132, 162 that can accommodate a portion of the user's hand (e.g., the palm or fingers) while gripping the handle 102.

In this example, grip accessory 130 may form a C-shaped cross-section and the groove 132 may span an arc of approximately 120-180 degrees. The grip accessory 160 may include a groove 162 with a relatively more compact profile than the groove 132, including dimensions proportional to the smaller size of the grip accessory 160. In some aspects, groove 162 may extend across approximately 90-150 degrees of the circumference of grip accessory 160, covering a smaller arc span than groove 132. Groove 162 may be on a lateral portion of grip accessory 160 and oriented at an angle relative to the longitudinal axis of port 114 to accommodate a user's thumb or other fingers when gripping the handle 102.

Both groove 132 and groove 162 may include various surface features, such as raised ridges and/or textured areas, to help a user grip handle 102. Grip accessory 130, grip accessory 160, and/or any other grip accessory described herein may comprise rubber-like materials or include tactile surfaces helping assist a user with their grip during a procedure. Handle 102 may also include additional ergonomic features. For example, the hub 104 includes a curved portion 103 that may receive fingers (e.g., an index finger and/or a middle finger) of the user's hand.

Medical device 100 may use different grip accessories with different structures, provided they include compatible engagement features able to removably couple to mounting interfaces 108, 118 on the outer surface of handle 102. For example, medical device 100 may include a mounting interface 108 and a mounting interface 118 including a plurality of engagement features 109 and a plurality of engagement features 119, respectively. The grip accessory 130 and the grip accessory 160 may also include a plurality of engagement features 139 and a plurality of engagement features 169, corresponding to those on the mounting interface 108 and mounting interface 118, respectively.

Engagement features 109, 119, 139, and 169 or any engagement features described herein may include snap-fit or compression-fit mechanisms, such as protrusions and recesses, pins and openings, Velcro®, or any other suitable attachment features and mechanisms. For example, each of a protrusion or recess on the engagement feature 109 of mounting interface 108 may align with a corresponding protrusion or recess on engagement features 139 of the grip accessory 130 to removably couple grip accessory 130. The engagement features 109, 139 may be oriented substantially parallel to each other and/or substantially transverse to the central longitudinal axis of the handle 102. For the engagement features discussed herein, the configuration of the engagement features may vary depending on factors such as the material properties of hub 104 and grip accessory 130, the expected forces during use, and the need for repeated attachment and detachment. The position of mounting interface 108 and mounting interface 118 on handle 102 will be described below.

The first mounting interface 108 may be on the outer surface of hub 104, opposite both port 114 and curved portion 103, and proximate vent 120. The mounting interface 108 may span an arc along hub 104 of handle 102, for example, extending to the widened portion of hub 104. The second mounting interface 118 may be positioned on the outer surface of the port 114. In some aspects, the second mounting interface 118 may cover a smaller portion of the outer surface of the port 114 compared to the first mounting interface 108, resulting in correspondingly less of the plurality of engagement features 119. In some aspects, engagement features 119 and mounting interface 118 are positioned at the distal portion of port 114 or alternatively, positioned closer to the hub 104 of handle 102 depending on user preferences or grip accessories.

In a fully assembled configuration, grip accessory 130 may be releasably coupled to the mounting interface 108 on hub 104 via engagement features 139, which align with corresponding engagement features 109 on mounting interface 108. For example, engagement features 139 align to mounting interface 108, such that engagement features 139 also span an arc. Similarly, grip accessory 160 may be releasably coupled to mounting interface 118 on port 114 via engagement features 169, which align with corresponding engagement features 119 on mounting interface 118.

FIG. 2 depicts another exemplary medical device 200 according to aspects of the disclosure. Medical device 200 may include any of the features of medical device 100, except as described herein. Optionally, medical device 200 may be configured to receive a medical instrument such as a scope to deliver medical instruments to access internal areas of a subject's body. The medical device 200 includes a handle 202 that may be coupled to or integral with a sheath 201. Thus, for example, handle 202 may be fluid communication with a channel of sheath 101.

The handle 202 includes a hub 204, similar to hub 104, that may define a proximal opening 207 to slidably receive a medical instrument such as a scope (e.g., ureteroscope, endoscope, hysteroscope, bronchoscope, cystoscope, or other scope or similar device) into a lumen defined by handle 102 (e.g., a lumen of hub 104). The proximal opening 207 may include a seal 250 configured to maintain a position of a medical instrument relative to the medical device 200, forming a substantially airtight around the medical instrument received therein. The handle 202 also includes a port 214, similar to port 214, in fluid communication with the lumen of hub 204. Handle 202 may include features that help the user to control or adjust pressure when port 214 is coupled to a vacuum source. As shown in FIG. 2, hub 204 includes a vent 220, similar to vent 120, including an aperture and a slider 222 configured to cover and uncover the aperture of vent 220.

The handle 202 may include ergonomic features similar to those described for the handle 102. For example, handle 202 may include a grip accessory 230 and a grip accessory 260, similar to grip accessory 130, 160, except as described herein. For example, grip accessory 230 and grip accessory 260 may comprise a semi-circular or ring-like structure including an opening 232 and an opening 262, respectively. Opening 232 and opening 262 may accommodate a portion of the user's hand or fingers while gripping handle 202. Handle 202 may also include additional ergonomic features. For example, the hub 204 includes a curved portion 203, similar to curved portion 103, which may receive fingers (e.g., an index finger and/or a middle finger) of the user's hand.

Medical device 200 may include a mounting interface 208 and a mounting interface 218, similar to mounting interface 108, 118, including a plurality of engagement features 209 and a plurality of engagement features 219, respectively. Similarly, grip accessory 230 and grip accessory 260 include a plurality of engagement features 239 and a plurality of engagement features 269, which may align with corresponding engagement features 209 and 219, respectively. The position of mounting interface 208 and the mounting interface 218 on the handle 202 will now be described.

Similar to the example of FIG. 1, mounting interface 208 may be on the outer surface of hub 204, opposite curved portion 203 and proximate the vent 220. As shown in FIG. 2, mounting interface 208 and engagement features 209 may extend along a distal portion of hub 204, terminating near the widened portion of handle 202 in a pattern matching the single row of engagement features 239 on the inner surface of the grip accessory 230. The mounting interface 218 may be positioned on the outer surface of the port 214. In some aspects, the mounting interface 218 may extend along a length of the outer surface of the port 214 comparable to the length of mounting interface 208 on the outer surface of hub 204. The fully assembled configuration of FIG. 2 may be similar to that of FIG. 1, including grip accessory 230 releasably coupled to hub 204 and grip accessory 260 releasably coupled to port 214.

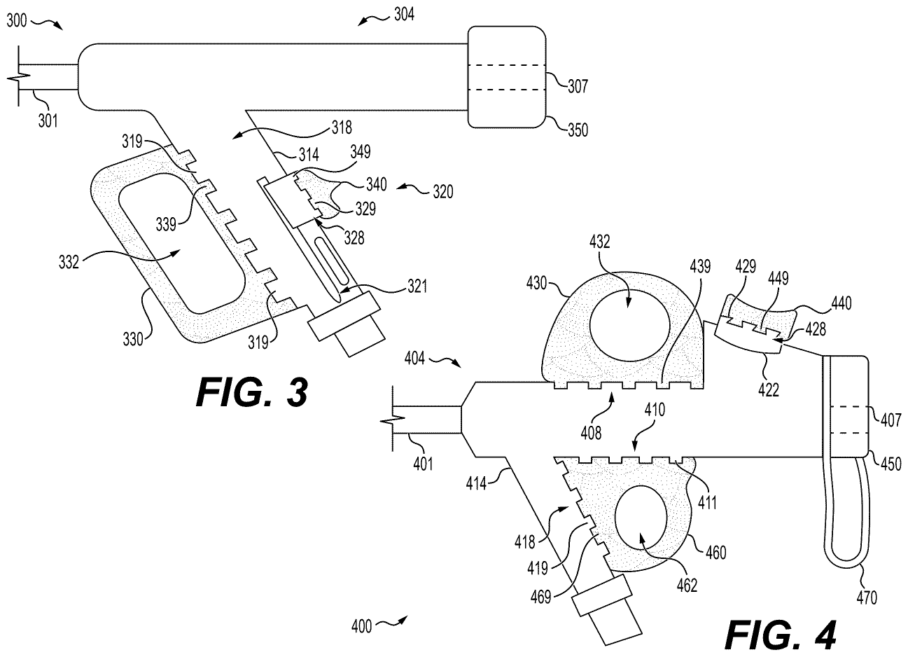

FIG. 3 depicts another exemplary medical device 300 according to aspects of the disclosure. Medical device 300 may include any of the features of medical device 100 and/or medical device 200, except as described herein. Optionally, medical device 300 may be configured to receive a medical instrument such as a scope to deliver medical instruments to access internal areas of a subject's body. Medical device 300 may be an aspiration sheath similar to the other medical devices described herein but with handle features configured to receive different grip accessories, discussed further below.

Handle 302 includes a hub 304, similar to hub 104 or 204, that may define a proximal opening 307 to slidably receive a medical instrument such as a scope (e.g., ureteroscope, endoscope, hysteroscope, bronchoscope, cystoscope, or other scope or similar device) into a lumen defined by the handle 302 (e.g., a lumen of hub 304). The proximal opening 307 may include a seal 350 configured to maintain a position of a medical instrument relative to the medical device 300, forming a substantially airtight and/or watertight seal around the medical instrument received therein. In some aspects, hub 304 may have different features than hub 104 or 204. For example, hub 304 may have a narrower profile and lack a widened portion (e.g., compared to hub 104 and 204).

The handle 302 also includes a port 314, similar to port 114 or 214, in fluid communication with the lumen of hub 304. Handle 302 may include features that help the user to control or adjust pressure when coupled to a vacuum source. As shown in FIG. 3, port 314 includes a vent 320, similar to vent 120 and/or 220, including an aperture and a slider 322 configured to cover and uncover the aperture of vent 320. The slider 322 may be coupled to port 314 via a slot 321 or cavity on the outer surface of port 314. As described in more detail below, slider 322 of vent 320 may be movably coupled to port 314, including ergonomic features to help move slider 322.

Handle 302 may include ergonomic features similar to those described for handle 102 and/or 202. As shown in FIG. 3, port 314 may include the ergonomic features of handle 302, such as mounting interfaces that receive grip accessories. Handle 302 may include a grip accessory 330 similar to grip accessory 230 and a grip accessory 340. Grip accessory 340 may have a generally conical shape that tapers from a wider base to a narrower tip. The shape of grip accessory 340 may provide a surface for the user's thumb and/or other fingers to engage with while adjusting the position of slider 322. Alternatively, grip accessory 340 may feature an asymmetric taper, where one lateral side of the cone may have a steeper angle than the other side.

Grip accessory 330 may have a semi-rectangular structure with rounded corners and an opening 332. Grip accessory 330 may be elongated such that it can extend further along the length of port 314. Correspondingly, opening 332 may be elongated and longer compared to openings shown in FIGS. 1 and 2. The elongated configuration of grip accessory 330 and opening 332 may help users position multiple fingers or their palm within grip accessory 330 when gripping the handle 302.

Medical device 300 uses grip accessories with different structures, each including compatible engagement features configured to removably couple to mounting interfaces of handle 302. For example, handle 302 may include a mounting interface 318, similar to mounting interface 118 and/or 218, as well as a mounting interface 328. Mounting interfaces 308 and 328 may include a plurality of engagement features 309 and 329, respectively. Similarly, grip accessory 330 and grip accessory 340 include a plurality of engagement features 239 and a plurality of engagement features 349, respectively, which align with corresponding engagement features 339 and 329.

As discussed, in this example, the ergonomic features of handle 302 may be integrated into port 314. Accordingly, mounting interface 318 may be on the outer surface of port 314. In some aspects, mounting interface 318 may extend along the length of the outer surface of port 314, providing ample area for the attachment of, for example, grip accessory 330. As shown in FIG. 3, slider 322 may include second mounting interface 328. The mounting interface 318 and mounting interface 328 may be located on lateral sides of port 314, with the two mounting interfaces positioned opposite one another.

In a fully assembled configuration, grip accessory 330 may be releasably coupled to first mounting interface 308 on port 314 via engagement features 339, which align with corresponding engagement features 309 on mounting interface 308. Similarly, grip accessory 340 may be releasably coupled to mounting interface 328 on slider 322 via engagement features 349, which align with corresponding engagement features 329 on mounting interface 328. In some aspects, this arrangement may help a user maintain their grip on port 314 of handle 302 while moving slider 322 to adjust airflow or suction without repositioning their hand.

FIG. 4 depicts another exemplary medical device 400 according to aspects of the disclosure. Medical device 400 may include any of the features of the medical devices discussed above, except as described herein. Optionally, medical device 400 may be configured to receive a medical instrument such as a scope to deliver medical instruments to access internal areas of a subject's body. Medical device 400 may be an aspiration sheath similar to the other medical devices described herein but with handle features configured to receive different grip accessories, discussed further below.

The handle 402 includes a hub 404 that may define a proximal opening 407 to slidably receive a medical instrument such as a scope (e.g., ureteroscope, endoscope, hysteroscope, bronchoscope, cystoscope, or other scope or similar device) into a lumen defined by handle 402 (e.g., a lumen of hub 404). The proximal opening 407 may include a seal 450 configured to maintain a position of a medical instrument relative to medical device 400, forming a substantially airtight and/or watertight seal around the medical instrument received therein. In some aspects, seal 450 may include a removable tether 470 to cover opening 407 when not in use.

The handle 402 also includes a port 414 in fluid communication with the lumen of hub 404. Handle 402 may include features that help the user to control or adjust pressure when coupled to a vacuum source. As shown in FIG. 4, hub 404 includes a vent 420 including an aperture and a slider 422 configured to cover and uncover the aperture of vent 420. As described in more detail below, slider 422 similar to slider 322, may be movably coupled to hub 404 including ergonomic features to help move slider 422.

The handle 402 may include ergonomic features similar to those described for any of the handles described above. In this example, FIG. 4 shows handle 402 may include three different grip accessories. For example, a grip accessory 440, similar to grip accessory 340. The shape of grip accessory 440 may provide a surface for the user's thumb or other fingers to engage with while adjusting the position of slider 422. Grip accessory 440 may have a crescent-like profile, with a curved outer surface.

The handle 402 may also include a grip accessory 430 and a grip accessory 460, similar to grip accessory 260, except as described herein. For example, grip accessory 430 and grip accessory 460 may comprise a semi-circular or ring-like structure including an opening 432 and an opening 462, respectively. Opening 432 and opening 462 may accommodate a portion of the user's hand (palm and/or fingers) while gripping handle 402. In some aspects, grip accessory 430 may have a substantially circular cross-section near the distal end of the hub 404 and transition to a more linear or flattened profile as it approaches a widened portion of handle 402.

Medical device 400 utilizes grip accessories with various structures, each featuring compatible engagement features configured to removably couple to the mounting interfaces on handle 402. For example, handle 402 may include a first mounting interface 408 and a second mounting interface 410, similar to mounting interface 208 and other mounting interfaces discussed above. Additionally, handle 402 may include a port mounting interface 418, similar to mounting interface 308, as well as a third mounting interface 428 positioned on slider 422, similar to mounting interface 328. The first mounting interface 408, second mounting interface 410, and third mounting interface 428 may each include a plurality of engagement features 409, 411, and 429, respectively. Similarly, the grip accessory 430, grip accessory 440, and grip accessory 460 may each include a plurality of engagement features 439, 449, and 469, respectively, which align with and correspond to the engagement features on the mounting interfaces 408, 410, and 428.

Similar to the example shown in FIG. 1, first mounting interface 408 may be located on the outer surface of hub 404, positioned proximate to vent 420, which includes slider 422 with third mounting interface 428 on its outer surface. As depicted in FIG. 4, mounting interface 408 and its engagement features 409 may extend along a distal portion of the hub 404, terminating near the widened portion of hub 404. Second mounting interface 410 may be positioned on the side of the hub 404 opposite the first mounting interface 408, aligned along the lateral axis of the hub 404.

The mounting interface 418 of port 414 may be positioned on the outer surface of the port 414. In some configurations, port mounting interface 418 may extend along the length of the outer surface of port 414 and terminate at a junction where port 414 meets hub 404. At this transition point, engagement features 411 of second mounting interface 410 may begin and extend along the outer surface of hub 404. This configuration may help grip accessory 460 span both interfaces, providing a continuous gripping surface. For example, grip accessory 460 may include engagement features 469 arranged in a V pattern, corresponding to engagement features 419 on port mounting interface 418 and engagement features 411 on second mounting interface 410. Alternatively, separate grip accessories may be independently attached to either the port mounting interface 418 or second mounting interface 410.

In a fully assembled configuration, the grip accessory 430 may be releasably coupled to the first mounting interface 408 on the hub 404. Similarly, the grip accessory 460 may be releasably coupled to the second mounting interface 410 and the port mounting interface 418, spanning both the hub 404 and port 414 for a continuous gripping surface. Additionally, the grip accessory 440 may be releasably coupled to the mounting interface 428 on the slider 422 via engagement features 449, which align with corresponding engagement features 429 on the mounting interface 428. In some aspects, this arrangement may help a user maintain their grip on port 414 and hub 404 while being able to move slider 422 to adjust airflow or suction without repositioning their hand.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical device comprising:
a handle comprising:
a hub defining a lumen and including a proximal opening configured to receive a medical instrument; and
a port configured to couple to a source of suction, the port being in fluid communication with the lumen of the hub;
wherein an outer surface of the hub or the port includes a mounting interface; and
a grip accessory configured to releasably couple to the mounting interface, wherein the grip accessory includes an opening or groove configured to receive a portion of a user's hand when the user grips the handle.

2. The medical device of claim 1, wherein the mounting interface is a first mounting interface and one of the hub or the port includes a second mounting interface.

3. The medical device of claim 2, wherein the grip accessory is a first grip accessory, and the medical device further comprises a second grip accessory configured to releasably couple to the second mounting interface.

4. The medical device of claim 2, wherein the grip accessory is configured to releasably couple to both the first mounting interface, and the second mounting interface.

5. The medical device of claim 2, wherein the hub includes the first mounting interface, and the port includes the second mounting interface.

6. The medical device of claim 1, wherein the mounting interface includes a plurality of engagement features configured to receive a plurality of engagement features of the grip accessory.

7. The medical device of claim 1, wherein the mounting interface spans an arc.

8. The medical device of claim 1, wherein the port or the hub includes a vent, and the mounting interface is proximate the vent.

9. The medical device of claim 8, wherein the vent includes a slider configured to cover and uncover an aperture of the port or the hub.

10. The medical device of claim 9, wherein the mounting interface is a first mounting interface, and the grip accessory is a first grip accessory, and wherein the slider includes a second mounting interface configured to releasably couple to a second grip accessory.

11. The medical device of claim 2, wherein at least one of the hub or the port includes a second mounting interface proximate the first mounting interface.

12. The medical device of claim 2, wherein the hub includes the first mounting interface, and the second mounting interface.

13. The medical device of claim 2, wherein the port includes the first mounting interface, and the second mounting interface.

14. The medical device of claim 1, further comprising a sheath defining a channel in fluid communication with the lumen and the port of the handle.

15. A medical device comprising:
a handle comprising:
a hub defining a lumen and including a proximal opening configured to receive a medical instrument; and
a port configured to couple to a source of suction, the port being in fluid communication with the lumen of the hub;
wherein an outer surface of the hub or the port includes a first mounting interface and a second mounting interface; and
a first grip accessory configured to releasably couple to the first mounting interface; and
a second grip accessory configured to releasably couple to the second mounting interface,
wherein the first grip accessory and the second grip accessory include an opening or groove configured to receive a portion of a user's hand when the user grips the handle.

16. The medical device of claim 15, wherein the handle further comprises a vent including a slider configured to cover and uncover an aperture of the port or the hub, wherein the first mounting interface is proximate the vent.

17. The medical device of claim 15, wherein the first mounting interface is on the hub and spans an arc along the outer surface of the hub, wherein the second mounting interface is on the port and extends a length of the outer surface of the port.

18. The medical device of claim 17, wherein the first mounting interface includes a plurality of engagement features configured to releasably couple with corresponding engagement features on the first grip accessory.

19. A medical device comprising:
a handle comprising:
a hub defining a lumen and including a proximal opening configured to receive a medical instrument;
a port configured to couple to a source of suction, the port being in fluid communication with the lumen of the hub;
a vent including a slider configured to cover and uncover an aperture of the port or the hub;
wherein an outer surface of the port includes a first mounting interface, wherein an outer surface of the slider includes a second mounting interface; and
a first grip accessory configured to releasably couple to the first mounting interface; and
a second grip accessory configured to releasably couple to the second mounting interface.

20. The medical device of claim 19, wherein the hub includes a third mounting interface on the hub.
